(19) United States
(12) Patent Application Publication
(10) Pub. No.: US 2025/0115896 A1
(43) Pub. Date: Apr. 10, 2025

(54) SYSTEM AND METHOD FOR THE PRODUCTION OF RNA

(71) Applicant: QUANTOOM BIOSCIENCES S.A., Nivelles (BE)

(72) Inventors: Anne DE LAMOTTE, Liège (BE); Bastien MAIRESSE, Uccle (BE)

(21) Appl. No.: 18/912,416

(22) Filed: Oct. 10, 2024

Related U.S. Application Data
(63) Continuation of application No. PCT/EP2023/059819, filed on Apr. 14, 2023.
(60) Provisional application No. 63/331,555, filed on Apr. 15, 2022.
(60) Provisional application No. 63/411,471, filed on Sep. 29, 2022.

(30) Foreign Application Priority Data
Apr. 20, 2022 (BE) BE2022/5293

(51) Int. Cl. C12N 15/10; C12N 9/12

(52) U.S. Cl. CPC C12N 15/10; C12N 9/1247

(57) ABSTRACT

The current application relates to a system for producing RNA, said system comprising an in vitro transcription (IVT) unit and a processing unit for downstream processing of said compound, wherein the IVT unit comprises a plurality of chambers, wherein each of the plurality of chambers is configured to receive one or more IVT reagents and to execute an IVT reaction based on a DNA template, thereby obtaining a plurality of RNA molecules; and wherein the processing unit comprises a purification device for performing at least one downstream purification step of said plurality of RNA molecules, said purification device comprising one or more sample holders, said sample holders are suited to receive a container holding a liquid medium comprising RNA molecules and magnetic particles, and wherein a magnet is positioned at each sample, said magnet unit is configured to capture or to introduce a movement of said magnetic particles and wherein the sample holders are configured to perform a mechanical motion, such that the magnetic particles are mixed with the liquid. The current application also discloses a method for the production of RNA, more preferably mRNA.

### CROSS-REFERENCE

This application is a Continuation of International Application No. PCT/EP2023/059819, filed Apr. 14, 2023, which claims the benefit of U.S. Provisional Application No. 63/331,555, filed Apr. 15, 2022, U.S. Provisional Application No. 63/411,471, filed Sep. 29, 2022, and Belgium Application No. BE2022/5293, filed Apr. 20, 2022, each of which is incorporated herein by reference in its entirety.

### FIELD OF THE APPLICATION

The present application relates to a system and method for the production of RNA, allowing efficient large scale RNA production for further downstream application such as the production of RNA vaccines.

### BACKGROUND

Therapeutic nucleic acids including RNA molecules represent an emerging class of drugs. RNA-based therapeutics include mRNA molecules encoding antigens for use as vaccines. In addition, it is envisioned to use RNA molecules for replacement therapies, e.g. providing missing proteins such as growth factors or enzymes to patients. Furthermore, the therapeutic use of RNA molecules for genome editing (e.g. CRISPR/Cas9 guide RNAs) is considered. Accordingly, RNA-based therapeutics with the use in immunotherapy, gene therapy and vaccination belong to the most promising and quickly developing therapeutic fields in modern medicine.

Currently established manufacturing processes for RNA molecules implement many separate manufacturing steps. Particularly, the respective manufacturing steps are performed by several different devices. In addition, manufacturing of RNA requires a large degree of manual handling in a GMP-regulated laboratory executed by well-trained technical staff. As a consequence, current established manufacturing processes are time consuming, cost intensive, and require a lot of laboratory space and laboratory equipment.

Automated analyzer systems are known from U.S. Pat. No. 9,372,156B2, CN211402409 and CN113504380, however, these are not suitable for in vitro transcription. A system for performing IVT that uses 2D purification of the mRNA is known from WO2021030271. Nonetheless, 2D purification presents major drawbacks, and alternative purification methods that employ magnetic particles, such as those disclosed in WO2016077294A1 and US2013302810A, could circumvent these problems.

The current invention provides a solution to at least some of these problems, by providing an efficient automated RNA production and purification system.

### SUMMARY

The present disclosure and embodiments thereof serve to provide a solution to one or more of above-mentioned disadvantages. To this end, the present disclosure provides an automated system and method for the production of RNA. The system and method allows for a fast, efficient, and automated large scale production of RNA that complies with the quality requirements for use in therapeutics. Conventionally, in order to manage the production of a large amount of therapeutic products such as RNA, a considerable number of large instruments is needed. Compactness of the design and the amount of support resources has however become an important issue. The COVID-19 pandemic and the following shortage in COVID-19 RNA vaccines learned that there is a considerable need for mobile, easily to be transported and easy to operate solutions for the production of RNA and RNA based therapeutics.

By combining certain steps in an efficient and ingenious manner, thereby optimally making use of the available space, the footprint of the current system is kept low. As a consequence, the system can easily be transported, even to remote places. In addition, because of the highly automated methodology, the system is easy to be operated and doesn't require highly trained or highly technical staff. The present method and system are (almost) devoid of manual handling, thereby also considerably reducing contamination risk and the risk of degradation of the end product. Due to the (semi) continuous process, large amounts of RNA can be produced in a short amount of time.

### DESCRIPTION OF FIGURES

FIG. 1 shows a representation of a system according to embodiments of the disclosure.

FIG. 2 shows a representation of an IVT unit according to embodiments of the disclosure.

FIG. 3A and FIG. 3B show representations of purification devices according to embodiments of the disclosure.

FIG. 4A and FIG. 4B presents the principle of capturing magnetic beads using the magnetic unit. In FIG. 4A the beads are free and in FIG. 4B the beads are captured.

FIG. 5 shows a representation of a sample holder according to an embodiment of the disclosure.

FIG. 6 shows a representation of the sample holder according to another embodiment of the disclosure.

FIG. 7 presents a schematic representation of an embodiment of the downstream processing unit, having two adjacent purification devices.

FIG. 8 presents a schematic representation of an embodiment of the downstream processing unit, having two nested devices.

FIG. 9 schematically presents a robotic arm according to an embodiment of the disclosure.

FIG. 10 shows a representation of a cabinet according to an embodiment of the disclosure.

FIG. 11 shows a representation of a cabinet fitting in a transport container according to an embodiment of the disclosure.

### DETAILED DESCRIPTION

US 2021/0261897 describes a bioreactor for in vitro transcription. However, the system is only suited for executing a first step of the RNA production chain, and does not provide a fully integrated system for the RNA production. Moreover, the bioreactor described herein does not allow a (semi-)continuous production of RNA.

The present disclosure aims to resolve at least some of the problems and disadvantages mentioned above. Further, the present disclosure describes a system and method for large scale, (semi)continuous RNA production.

### Definitions

Unless otherwise defined, all terms used, including technical and scientific terms, have the meaning as commonly understood by one of ordinary skill in the art. By means of further guidance, term definitions are included to better appreciate the teaching of the present application.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed application. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that embodiments described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

As used herein the term “magnetic particle” and variations thereof is intended to denote a particle with a magnetic, e.g., paramagnetic or superparamagnetic, core coated with at least one material having a surface to which a compound of interest can reversibly bind.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used, including technical and scientific terms, have the meaning as commonly understood by one of ordinary skill in the art. By means of further guidance, definitions for the terms used in the description are included to better appreciate the teaching of the present application. The terms or definitions used herein are provided solely to aid in the understanding of the application.

Reference throughout this specification to “one embodiment” or “an embodiment” means that a particular feature, structure or characteristic described in connection with the embodiment is included in at least one embodiment of the present application. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the application, and form different embodiments, as would be understood by those in the art. For example, in the following claims, any of the claimed embodiments can be used in any combination.

### Systems and Components of Said System

In a first aspect, the current disclosure discusses a system for the production of RNA. The term RNA or RNA molecules encompasses long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Trans amplifying mRNA, RNA oligonucleotides, antisense oligonucleotides, small interfering RNA (siRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), and a Piwi-interacting RNA (piRNA). In some embodiments, said RNA comprises modified RNA molecules. In some embodiments, the modification of RNA molecule comprises chemical modifications comprising backbone modifications as well as sugar modifications or base modifications. In this context, a modified RNA molecule as defined herein comprises nucleotide analogues/modifications, e.g. backbone modifications, sugar modifications or base modifications. A backbone modification in connection with the present disclosure is a modification, in which phosphates of the backbone of the nucleotides contained in an RNA molecule are chemically modified. A sugar modification in connection with the present disclosure is a chemical modification of the sugar of the nucleotides of the RNA molecule. Furthermore, a base modification in connection with the present disclosure is a chemical modification of the base moiety of the nucleotides of the RNA molecule. In this context, nucleotide analogues or modifications are selected from nucleotide analogues, which are applicable for transcription and/or translation. In further embodiments, the modified RNA comprises nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

In an embodiment, a system for producing RNA is disclosed, said system comprising an in vitro transcription (IVT) unit and a processing unit for downstream processing of said RNA molecules, wherein the IVT unit comprises a plurality of chambers, wherein each of the plurality of chambers is configured to receive one or more IVT reagents and to execute an IVT reaction based on a DNA template, thereby obtaining a plurality of RNA molecules; and wherein the processing unit comprises a purification device for performing at least one downstream purification step of said plurality of RNA molecules, said purification device comprising one or more sample holders, said sample holders are suited to receive a container holding a liquid medium comprising RNA molecules and magnetic particles, and wherein a magnet unit is positioned at each sample holder, said magnet unit is configured to capture or to introduce a movement of said magnetic particles and wherein the sample holders are configured to perform a mechanical motion, such that the magnetic particles are mixed with the liquid. Advantageously, the system as disclosed herein allows automation of the IVT of RNA molecule and downstream purification of said RNA molecules while executing the procedures continuously and with high precision without requiring human intervention. The production protocols can be directly implemented on the system as disclosed herein without any process scale-up as the device mimics small-scale manual operations executed in a laboratory.

The IVT unit is to be understood as that part of said system that accommodates an IVT reaction starting from a DNA template, by means of a DNA dependent RNA polymerase. In some embodiments, the DNA dependent RNA polymerases comprise at least one of a T7 RNA polymerase, a T3 RNA polymerase, a SP6 RNA polymerase, a RNA polymerase I, a RNA polymerase II, a RNA polymerase III, a RNA polymerase IV, a RNA polymerase V, and a single subunit RNA polymerase. The DNA template for RNA in vitro RNA transcription may be obtained by cloning of a nucleic acid, in particular cDNA corresponding to the respective RNA to be in vitro transcribed, and introducing it into an appropriate vector for RNA in vitro transcription, for example in plasmid circular plasmid DNA. The cDNA may be obtained by reverse transcription of mRNA or chemical synthesis. Moreover, the DNA template for in vitro RNA synthesis may also be obtained by gene synthesis.

In some embodiments, the DNA template relates to a DNA molecule comprising a nucleic acid sequence encoding the RNA sequence. The template DNA is used as a template for RNA in vitro transcription in order to produce the RNA encoded by the template DNA. Therefore, the template DNA comprises all elements necessary for RNA in vitro transcription, particularly a promoter element for binding of a DNA dependent RNA polymerase as e.g. T3, T7 and SP6 RNA polymerases 5′ of the DNA sequence encoding the target RNA sequence. The poly(A) tail can be either encoded into the DNA template or added enzymatically to RNA in a separate step after in vitro transcription. In some embodiments, the template DNA comprises primer binding sites 5′ and/or 3′ of the DNA sequence encoding the target RNA sequence to determine the identity of the DNA sequence encoding the target RNA sequence e.g. by PCR or DNA sequencing. In some embodiments, the DNA template comprises a DNA vector, such as a plasmid DNA, which comprises a nucleic acid sequence encoding the RNA sequence. In some embodiments, the DNA template comprises a linear or a circular DNA molecule.

In some embodiments, the chambers of the IVT unit are housed in a cartridge. In a further embodiment, the cartridge comprises between 2 and 48 chambers.

In some embodiments, the cartridge comprises at least 2 chambers, 3 chambers, 4 chambers, 5 chambers, 6 chambers, 7 chambers, 8 chambers, 9 chambers, 10 chambers, 11 chambers, 12 chambers, 13 chambers, 14 chambers, 15 chambers, 16 chambers, 17 chambers, 18 chambers, 19 chambers, 20 chambers, 21 chambers, 22 chambers, 23 chambers, 24 chambers, 25 chambers, 26 chambers, 27 chambers, 28 chambers, 29 chambers, 30 chambers, 31 chambers, 32 chambers, 33 chambers, 34 chambers, 35 chambers, 36 chambers, 37 chambers, 38 chambers, 39 chambers, 40 chambers, 41 chambers, 42 chambers, 43 chambers, 44 chambers, 45 chambers, 46 chambers, 47 chambers, 48 chambers, 49 chambers, or 50 chambers. In some embodiments, the cartridge comprises at least about 60 chambers, about 70 chambers, about 80 chambers, about 90 chambers, about 100 chambers, about 110 chambers, about 120 chambers, about 130 chambers, about 140 chambers, about 150 chambers, about 160 chambers, about 170 chambers, about 180 chambers, about 190 chambers, or about 200 chambers.

In an embodiment, the IVT unit comprises 1 cartridge, 2 cartridges, 3 cartridges, 4 cartridges, 5 cartridges, 6 cartridges, 7 cartridges, 8 cartridges, 9 cartridges, or 10 cartridges. In another embodiment, said IVT unit comprises between 1 and 10 cartridges, between 1 and 20 cartridges, between 1 and 30 cartridges or between 1 and 40 cartridges, 1 and 50 cartridges, 1 and 60 cartridges, 1 and 70 cartridges, 1 and 80 cartridges, 1 and 90 cartridges, or 1 and 100 cartridges.

In an embodiment, the chamber of the IVT unit comprises at least three flat polygonal surfaces. In another embodiment, the chamber comprises one, two, three, four, five, six, seven, or eight flat polygonal surfaces, preferably six flat polygonal surfaces. Advantageously, the design of the chamber allows for it to be equipped with a sensor and/or a probe

In an embodiment, the cross-section of the chamber is polygonal, such as trigonal, tetragonal, pentagonal, or hexagonal. In a preferred embodiment, the cross-section of the chamber is hexagonal. A plurality of hexagonal chambers, eg combined in a cartridge, may create a honey-comb shaped structure. Without wishing to be bound to theory, the hexagonal shape is known in geometry to best fill a plane with equal size units without leaving out unused space. Moreover, hexagonal packing also minimizes the perimeter for a given area because of its 120-degree angles, and thus the hexagonal shape of the chamber ensures less use of raw material.

In an embodiment, the open end of the chamber is configured to receive a removable lid for at least partially closing said open end. The lid prevents unwanted components from entering said chamber (for example, RNases, microbial contamination or other degrading compounds or organisms) and shields the content of said chamber from the outer environment. In an embodiment, said lid also prevents evaporation of the content of said chamber. In some embodiments, the chamber comprises the lid to limit exchange with the environment. In some embodiments, the lid of the chamber is removable. In some embodiments, the lid of the chamber is not removable. Alternatively, the chamber is uncovered.

In some embodiments, the lid can prevent excessive water evaporation and loss of other critical volatile components. In some embodiments, the lid can prevent oxidation of the reagents or any components. In some embodiments, the lid can provide with protection from light (if needed). In some embodiments, the lid prevents contamination from any other potential chemical compound.

In some embodiments, the lid comprises at least one opening for filling, draining and sampling. In some embodiments, the at least one opening is positioned on the top of the lid.

In some embodiments, the bottom of the chamber is rounded. In other embodiments, the bottom of the chamber is flat or pointed.

In some embodiments, the chamber is removable from the cartridge. In some embodiments, the chamber is not removable from the cartridge.

System according to any of the previous claims, wherein the chambers comprise a volume of about 0.1 mL to 500 mL.

In some embodiments, the chamber comprises a volume of at least about 0.1 ml, about 0.3 ml, about 0.5 ml, about 1 ml, about 1.5 ml, about 2 ml, about 2.5 ml, about 3 ml, about 4 ml, about 5 ml, about 6 ml, about 7 ml, about 8 ml, about 9 ml, about 10 ml, about 12 ml, about 15 ml, about 17 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, or about 100 ml. In some embodiments, the chamber comprises a volume of not more than about 100 ml, not more than about 95 ml, not more than about 90 ml, not more than about 85 ml, not more than about 80 ml, not more than about 75 ml, not more than about 70 ml, not more than about 65 ml, not more than about 60 ml, not more than about 55 ml, not more than about 50 ml, not more than about 45 ml, not more than about 40 ml, not more than about 35 ml, not more than about 30 ml, not more than about 25 ml, not more than about 20 ml, not more than about 15 ml, not more than about 10 ml, not more than about 9 ml, not more than about 8 ml, not more than about 7 ml, not more than about 6 ml, not more than about 5 ml, not more than about 4 ml, not more than about 3 ml, not more than about 2 ml, not more than about 1 ml, not more than about 0.5 ml, not more than about 0.3 ml, or not more than about 0.1 ml. In some embodiments, the chamber comprises a volume of between about 1 ml to about 100 ml, between about 10 ml to 90 ml, between about 15 ml to about 80 ml, between about 20 ml to about 70 ml, between about 25 ml to about 60 ml or between about 30 ml to about 50 ml.

In some embodiments, the chamber comprises a volume of at least about 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, about 1000 ml, about 2000 ml, about 3000 ml, about 4000 ml, about 5000 ml, about 6000 ml, about 7000 ml, about 8000 ml, about 9000 ml, about 10000 ml, about 15000 ml, about 20000 ml, about 25000 ml, about 30000 ml, about 35000 ml, about 40000 ml, about 45000 ml, or about 50000 ml. In some embodiments, the chamber comprises a volume of not more than about 50000 ml, not more than about 45000 ml, not more than about 40000 ml, not more than about 35000 ml, not more than about 30000 ml, not more than about 25000 ml, not more than about 20000 ml, not more than about 15000 ml, not more than about 10000 ml, not more than about 9000 ml, not more than about 8000 ml, not more than about 7000 ml, not more than about 6000 ml, not more than about 5000 ml, not more than about 4000 ml, not more than about 3000 ml, not more than about 2000 ml, not more than about 1000 ml, not more than about 950 ml, not more than about 900 ml, not more than about 850 ml, not more than about 800 ml, not more than about 750 ml, not more than about 700 ml, not more than about 650 ml, not more than about 600 ml, not more than about 550 ml, not more than about 500 ml, not more than about 450 ml, not more than about 400 ml, not more than about 350 ml, not more than about 300 ml, not more than about 250 ml, not more than about 200 ml, not more than about 150 ml. In some embodiments, the chamber comprises a volume of between about 150 ml to about 50000 ml, between about 200 ml to 45000 ml, between about 250 ml to about 40000 ml, between about 300 ml to about 35000 ml, between about 350 ml to about 30000 ml, between about 400 ml to about 25000 ml, between about 450 ml to about 20000 ml, between about 500 ml to about 15000 ml, between about 550 ml to about 10000 ml, between about 600 ml to about 9000 ml, between about 650 ml to 8000 ml, between about 700 ml to about 7000 ml, between about 750 ml to about 6000 ml, between about 800 ml to about 5000 ml, between about 850 ml to about 4000 ml, between about 900 ml to about 3000 ml, between about 950 ml to about 2000 ml or between about 1000 ml to about 1500 ml.

In some embodiments, the IVT unit comprises at least one handling apparatus configured for dispensing and/or removing a reagent, a reagent mixture, or a liquid in said chambers. Without being limitative the liquid dispensed and/or removed can be deionized water, purified water, a buffer solution, a cleaning solution, a reagent, an enzyme, a DNA template, a transcription buffer, a solution comprising nucleotide triphosphates (NTPs), an RNase inhibitor, or an RNA polymerase, or a combination thereof, or a waste product. Non-limitative examples of buffers are magnesium acetate, HEPES, Tris, or Tris HCl. In a preferred embodiment, the liquid dispensed and/or removed is an IVT premix comprising water, RNA polymerase buffer, linearized DNA template, and dNTPs. In a further preferred embodiment, the IVT premix comprises water, T7 polymerase buffer, linearized DNA template, and dNTPs.

The handling apparatuses of the IVT unit can be an injector or a robotic arm provided with one or more nozzles, needles, and/or tips. Any injectors and robotic arms that are known in the art and are capable of dispensing and/or removing a component or a liquid can be used with the system as disclosed herein. Non-limiting examples of handling apparatuses are syringe pumps, vacuum pumps, peristaltic pumps, centrifugal pumps, or a combination thereof. In some embodiments, the pipettes, micropipettes, needles or tips are removable and/or single-use. In other embodiments, the pipettes, micropipettes, needles or tips are not removable.

Alternatively, the dispensing and/or removing of components is done by the handling apparatuses directly via the tubing they are provided with.

In an embodiment, the IVT unit as disclosed herein comprises a robotic arm. In another embodiment, the IVT unit comprises one or more injectors. In yet another embodiment the IVT unit comprises one or more pumps. In yet another embodiment, the IVT unit comprises a robotic arm and one or more injectors. In yet another embodiment, the IVT unit comprises a robotic arm and one or more pumps. In a preferred embodiment, the IVT unit comprises a robotic arm, one or more injectors, and one or more pumps.

The handling apparatuses of the IVT unit are configured to perform movements. In some embodiments of the IVT unit, said movement is a vertical, horizontal, centrifugal, or a 3D movement. In an embodiment, said handling apparatuses are controlled by motor units, preferably electric motors.

In some embodiments, the IVT unit comprises a control system arranged and adapted to control the dispensing and/or removing of a reagent, reagent mixture or liquid by said handling apparatuses. Based on the input information, such as sensor data and predefined algorithms, the control system regulates the performance of the handling apparatuses.

In an embodiment of the IVT unit, the handling apparatuses are adapted to dispense an amount of an IVT premix and/or one or more enzymes in said chambers.

In an embodiment, the system comprises a storage unit for storing one or more reagents, said storage unit can be cooled to a temperature below 10° C. In an embodiment, one reagent, two reagents, three reagents, four reagents, five reagents, six reagents, seven reagents, eight reagents, nine reagents, or ten reagents, preferably four reagents are stored in said storage unit. The reagents can be selected from water, T7 reaction buffer, linearized DNA template, and NTPs but are not limited to these.

In some embodiments, the storage unit can be cooled below 10° C., 9° C., 8° C., 7° C., 6° C., 5° C., 4° C., 3° C. or 1° C., preferably the cooled storage is at 4° C.

The storage unit is in fluid connection with a pump system, such as a peristaltic pump or a syringe pump. In additional embodiments, the pump comprises a vacuum pump, a peristaltic pump, a centrifugal pump or combination thereof.

In an embodiment said fluid connection is via at least one tubing. In some embodiments the tubing comprises, and is preferably made of, a material selected from the group consisting of polyethylene (PE), nylon, urethane, copper, stainless steel, aluminum, polyvinyl chloride (PVC), polypropylene (PP), polyurethane (PU), vinyl, polyvinylidene fluoride (PVDF), fiberglass, glass, rubber, and combinations thereof. In some embodiments, the tubing is chemical resistant.

In an embodiment, the pump system is configured to provide a desired amount of one or more reagents for preparing an IVT premix to an IVT premix container.

In an embodiment, the system as disclosed herein comprises heating apparatuses for heating the IVT premix container. In an embodiment, the IVT premix container is heated at a temperature of between 35° C. and 55° C., preferably between 35° C. and 50° C., 35° C. and 45° C., 35° C. and 40° C., or 35° C. and 37° C. Alternatively, the IVT premix container is heated at a temperature of between 37° C. and 55° C., preferably between 40° C. and, 45° C. and 55° C. or 40 and 55° C. In a preferred embodiment, the premix container is heated at 37° C.

The system comprises in some embodiments an intermediate vessel for pooling the content of a finalized IVT reaction from the plurality of chambers. In some embodiments, the intermediate storage vessel is in fluid communication with the chambers. In other embodiments, the intermediate storage vessel is not in fluid communication with the chamber. In some embodiments, the intermediate storage vessel is configured to store the RNA molecules produced by IVT. In some embodiments, the intermediate storage vessel is configured to detect contamination of the RNA molecules. In some embodiments, the intermediate storage vessel contains means to perform certain chemical, physical or mechanical treatment of the RNA molecules and/or the undesired residuals—for example, binding of the product, partial elimination of residuals, or adjustment of pH value.

In an embodiment, the purification device of the processing unit disclosed herein comprises a sample plate, said sample plate comprises a base portion, and sample holders are provided on said base portion. The purification device allows for separation and/or purification of the RNA molecules that result from the IVT reaction, hance performing the downstream purification of RNA molecules in an automated way.

In an embodiment, each sample holder of the purification device is configured to perform a mechanical motion independently from the remaining sample holders. This allows for the execution of distinct steps of the separation and/or purification process in each sample holder. In other embodiments, the sample holders are performing simultaneous motions.

In an embodiment, the mechanical motioning of the sample holders is driven by a motor unit or electromagnetic unit. In a preferred embodiment, an electric motor is used. In another embodiment, a plurality of electromagnets is powered in sequence to generate the motion of the sample holder. In another embodiment, the mechanical motioning of the sample holders is driven by a shaker unit. The mechanical motion of the sample holders can be a rotation around an axis of said sample holder or a shaking or a vibrating motion. The mechanical motion of the sample holders can be activated and deactivated. The purpose of the mechanical motioning includes but is not limited to, homogenization of the mixture formed by the sample and the buffers for separation and/or purification, and/or facilitation of the contact between the magnetic particles and the sample.

In an embodiment, the sample holders can be positioned on top of the sample plate or are positioned in pockets or recesses of said sample plate.

In an alternative embodiment, the purification device does not comprise a sample plate, but comprises one or more sample holders present in said system.

In an embodiment, the magnet unit of each sample holder comprises a permanent magnet, a temporary magnet or an electromagnet, preferably a permanent magnet. When an electromagnet is used, the magnetic field can be quickly changed by controlling the amount of electric current. In another embodiment each magnet unit comprises an array of magnets where each magnet can be a permanent magnet, a temporary magnet or an electromagnet.

In a preferred embodiment, the magnet unit is positioned along a side portion of said sample holder and extends above said sample holder. In another embodiment, the magnet unit is positioned around the sample holder.

In another embodiment, the sample holder is equipped with at least two, three, four, five, six, seven, eight, nine, or ten magnet units. In an embodiment, said magnet units are positioned along different side portions of said sample holder. Alternatively, all the magnets are positioned along the same side portion of said sample holder.

In an embodiment, the magnet unit is the same length as the sample holder, or of a smaller length. In a particularly preferred embodiment, the magnet unit is rod-like and at least ⅕th of the length of the magnet unit extends above the sample holder. In another embodiment at least ¼, ⅓, ½, ⅖, 2/4, ⅔, 2/1, ⅗, ¾, 3/2, 3/1, ⅘, 4/3, 4/2, 4/1, 5/4, 5/3, 5/2 or 5/1 of the length of the magnet unit extends above the sample holder. In a preferred embodiment, the magnet unit has a height that is proportional to the height of the level of the liquid in the sample container. In a preferred embodiment, the height and thickness of said magnet unit are directly proportional to the sample container surface, in order to ensure a sufficiently large magnetic field for capturing magnetic particles. In another embodiment, the magnet unit extends at least the entire length of the sample container, preferably extending under the sample container.

In yet another embodiment the magnet is bar, horseshoe, disc, sphere, cylinder, or ring-shaped.

In an embodiment, the magnet unit is arranged in a housing, which is preferably open at the side facing the sample holder. Alternatively, the housing is positioned around the sample holder and contains multiple magnets. In another embodiment, the magnet unit is ring-shaped and fully surrounds the sample holder.

The housing can be fabricated from any suitable material known in the art, such as but not limited to polymers, thermoplastics, metals or metals alloys.

The magnet unit may be fixed or movable in position. For instance, the magnet unit may perform vertical or lateral movements. These movements can influence whether or not a the magnetic particles will be attracted to the magnet. If positioned to far, the magnetic particles will remain in the liquid.

In an embodiment, an adaptor is present in the sample holder, to adapt the size of said sample holder. The use of the adaptors allows for the device to be used with sample containers of various shapes and sizes. The adaptors can be made of any material suitable in the art such as PC (polycarbonate), PP (polypropylene), PAI (polyamide-imide) (e.g. Torlon), PI (polyimide) (e.g. Tecasint), PPS (polyphenylsulfide) (e.g. Tecatron), PPSU (polyphenylsulfone) (e.g. Tecason P), PSU (Polysulfone) (e.g. Tecason S), PEI (polyetherimid) (e.g. Tecapei), glass (e.g. borosilicate glass), technical ceramics (e.g. FRIDURIT®), Polyaryletherketone (e.g., Polyetheretherketon (PEEK)), thermoplastics (e.g. DuraForm® Pa or DuraForm® GF), metal or metal alloy.

The sample plate of the purification device disclosed herein is configured to rotate around an axis, preferably the central axis of said base portion. In a preferred embodiment, the sample plate is configured to rotate clockwise and counterclockwise. In another embodiment, the sample plate is configured to perform a linear movement. The rotation of the sample plate is driven by a motor unit, preferably an electric motor. Rotation of the sample plate allows the delivery of the sample container positioned in the sample holder, to different fixed dispensers, where a component or a liquid can be added or removed from the sample container.

In an alternative embodiment, the sample plate of the purification device disclosed herein is fixed or immobile, being unable to rotate around an axis.

The base portion of said sample plate can be rectangular, polygonal, circular, ellipsoidal, or annular. In an embodiment, the sample plate is circular and has a diameter of between 20 and 50 cm, more preferably between 20 and 40 cm, such as 35 cm or 30 cm. In another embodiment, the sample plate is rectangular. The sample plate can be fabricated from any suitable material known in the art such as polymers, metal, metal alloys, resins, or any nonmagnetic or paramagnetic material. Polymers include but are not limited to polystyrene, PVC, Perspex, or Lucite.

In an embodiment, the sample plate comprises a plurality of sample holders, and said sample holders are positioned at regular intervals along the circumference of said sample plate. In another embodiment, said plurality of sample holders are positioned at irregular intervals along the circumference of said sample plate. Alternatively, the sample holders are positioned along the sides of said sample plate.

In one embodiment, the plate can comprise one sample holder or a plurality of sample holders. The sample plate can comprise between 1 and 100, more preferably between 1 and 50 sample holders, more preferably between 1 and 20 sample holders, more preferably between 1 and 16 sample holders, more preferably 12 sample holders. For example, a sample plate can comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 sample holders.

In an embodiment, the sample holder has a shape that accommodates the shape of the sample container. In a preferred embodiment, the cross-section of the sample holder is hexagonal. It would be obvious, however, to one skilled in the art that the cross-section of the sample holder can have any shape, as described above, that can accommodate the shape of the sample container.

In some embodiments, the sample holder is removable from the sample plate. In some embodiments, the sample holder is a fixed part of the sample plate.

In an embodiment, the sample holders may comprise identification (ID) tags, for identifying a sample container when being present in said sample holder. The ID tags may comprise an RFID tag, a smart label, or a reader for reading an RFID tag or smart label. Smart labels include, but are not limited to QR codes and bar codes.

In some embodiments, the processing unit of the system comprises at least one handling apparatus configured for dispensing and/or removing a component or a liquid from a sample container present in a sample holder of said purification device. Said particle component can be a magnetic particle and said liquid can be deionized water, purified water, a buffered solution, a washing buffer, an elution buffer, a reagent, or a combination thereof, or a waste product. It should be apparent that said liquid is not limited to these.

The handling apparatuses used in conjunction with the purification device may comprise any apparatuses suitable in the art, such as an injector or a robotic arm, for instance provided with one or more nozzles, needles, and/or tips. Alternatively, the dispensing by the handling apparatuses is done directly via the tubing of the handling apparatuses. Any injector, pump, or robotic arm that is known in the art and is capable of dispensing and/or removing a component or a liquid can be used with the system as disclosed herein. Non-limiting examples of handling apparatuses are syringe pumps, vacuum pumps, peristaltic pumps, centrifugal pumps, or a combination thereof.

In an embodiment, the injectors and pumps are fixed dispensers placed in the vicinity of said sample plate and the sample container is brought to them by the rotation of the sample plate. In an embodiment, the robotic arm can move along three separate axes and is able to access the sample container independent of the rotation of the sample plate. In another embodiment, the robotic arm is positioned on the sample plate or in the center of said sample plate.

In an embodiment where the sample plate is fixed, handling of the samples, dispensing and/or removing a component or a liquid into the samples is performed by the robotic arm.

In an embodiment, at least one robotic arm is used in conjunction with the purification device. In another embodiment, one or more injectors are used in conjunction with the purification device. In yet another embodiment one or more pumps are used in conjunction with the purification device. In yet another embodiment, a robotic arm and one or more injectors are used in conjunction with the purification device. In yet another embodiment, a robotic arm and one or more pumps are used in conjunction with the purification device. In a preferred embodiment, a robotic arm, one or more injectors, and one or more pumps are used in conjunction with the purification device.

The handling apparatuses used in conjunction with the purification device are connected to one or more reagent storage, waste vessels, and/or harvest vessels. Any reagent storage, waste vessels, and harvest vessels known in the art can be used in conjunction with the system. Non-limiting examples include bags, vials, tubes, bottles, jars, or barrels.

In an embodiment, the handling apparatuses used in conjunction with the purification device are controlled by motor units, preferably electric motors, and are configured to perform movements. In some embodiments, said movement is a vertical, horizontal, centrifugal, or a 3D movement. In a preferred embodiment, the handling apparatuses are operatively coupled to at least one computer processor for controlling said handling apparatuses.

The system may further comprise a control system arranged and adapted to control the dispensing and/or removing of a component or liquid by said handling apparatuses used in conjunction with the purification device. Based on the input information, such as sample ID and sensor data, and predefined algorithms, the control system regulates the performance of the handling apparatuses.

In an embodiment, the system for the production of RNA molecules as disclosed herein comprises a plurality of purification devices as described above. In another embodiment, the system comprises two purification devices as described above. In a further embodiment said purification devices are positioned adjacent to each other. Alternatively, said purification devices are in a nested configuration. A multidevice system allows for the performance of multiple purification steps concomitant such that different stages of the separating and/or purifying can be performed at the same time. Moreover, a multidevice system allows for the continuous operation of said system and for processing a high number of samples.

The system may comprise at least one harvest vessel, for harvesting a compound of interest. In an embodiment, the system may comprise multiple harvest vessels. In another embodiment, the harvest vessel is positioned in an aperture of the sample plate of the device. In yet another embodiment, the harvest vessel is positioned in the system along the vicinity of the purification device. In yet another embodiment all the purification devices are foreseen with at least a harvest vessel. In yet another embodiment one purification device is provided with a harvest vessel while the other purification devices have no harvest vessel.

The system comprises one or more sample containers. Said sample containers are positioned in the sample holders of the purification device.

In an embodiment, the sample containers are disposable. In another embodiment, said containers can be reused multiple times.

In some embodiments, a material of the harvest vessel and/or sample container of the purification device comprises a material that is resistant to e.g. cleaning procedures (chemically resistant), extreme temperatures (e.g. denaturation of nucleic acids), extreme pH values (sanitization of the reactor with bases and acids, e.g. with NaOH), mechanical forces (e.g. frictions caused by magnetic particles), and/or corrosion. In additional embodiments, the material of the harvest vessel and/or sample container comprises a material of proper light permeation (transparent, translucent, or opaque) to a corresponding purpose. In some embodiments, the material of the harvest vessel and/or sample container comprises a material of proper gas permeation to a corresponding purpose. In further embodiments, the materials of the harvest vessel and/or sample container should be temperature conductive at working temperatures between 37° C. and 65° C. (e.g. W/(mK) values of at least 10, preferably at least 15). In some embodiments, the inner surface of the harvest vessel and/or sample container comprises a surface material that does not release unwanted compounds that may contaminate the end product. In further embodiments, the materials of the harvest vessel and/or sample container and/or the inner surface thereof are PC (polycarbonate), PP (polypropylene), PAI (polyamide-imide) (e.g. Torlon), PI (polyimide) (e.g. Tecasint), PPS (polyphenylsulfide) (e.g. Tecatron), PPSU (polyphenylsulfone) (e.g. Tecason P), PSU (Polysulfone) (e.g. Tecason S), PEI (polyetherimid) (e.g. Tecapei), glass (e.g. borosilicate glass), technical ceramics (e.g. FRIDURIT®), Polyaryletherketone (e.g., Polyetheretherketon (PEEK)), thermoplastics (e.g. DuraForm® Pa or DuraForm® GF), all of which being chemically resistant, pH resistant, and temperature resistant. In additional embodiments, the materials of the harvest vessel and/or sample container comprise a material for a single-use including, but not limited to, polyethylene terephthalate and other polyethylenes, polyvinyl acetate, polyvinyl chloride. In some embodiments, the materials of the harvest vessel and/or sample container comprise a material having resistance to sterilization process including steam treatment or ethylene oxide (EtO) exposure/gamma irradiation even before adding any reaction-related reagents. In some embodiments, the materials of the harvest vessel and/or sample container provide protection from light (if needed) for medium contained in the harvest vessel and/or sample container. In some embodiments, the harvest vessel and/or sample container are made of any nonmagnetic or paramagnetic material known in the art.

In some embodiments, the harvest vessel and/or sample container of the purification device are pie wedge shaped, regular or irregular polygon shaped, concave polygon shaped, convex polygon shaped, trigon shaped, quadrilateral polygon shaped, pentagon shaped, hexagon shaped, equilateral polygon shaped, equiangular polygon shaped, heptagon shaped, octagon shaped, nonagon shaped, decagon shaped, hendecagon shaped, dodecagon shaped, tridecagon shaped, tetradecagon shaped, pendedecagon shaped, hexdecagon shaped, heptdecagon shaped, octdecagon shaped, enneadecagon shaped, icosagon shaped, n-gon shaped, or elliptic shaped, preferably circular shaped.

In some embodiments, the harvest vessel and/or sample container of the purification device comprise a volume of at least about 0.1 ml, about 0.3 ml, about 0.5 ml, about 1 ml, about 1.5 ml, about 2 ml, about 2.5 ml, about 3 ml, about 4 ml, about 5 ml, about 6 ml, about 7 ml, about 8 ml, about 9 ml, about 10 ml, about 12 ml, about 15 ml, about 17 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, or about 100 ml. In some embodiments, the harvest vessel and/or sample container comprise a volume of not more than about 100 ml, not more than about 95 ml, not more than about 90 ml, not more than about 85 ml, not more than about 80 ml, not more than about 75 ml, not more than about 70 ml, not more than about 65 ml, not more than about 60 ml, not more than about 55 ml, not more than about 50 ml, not more than about 45 ml, not more than about 40 ml, not more than about 35 ml, not more than about 30 ml, not more than about 25 ml, not more than about 20 ml, not more than about 15 ml, not more than about 10 ml, not more than about 9 ml, not more than about 8 ml, not more than about 7 ml, not more than about 6 ml, not more than about 5 ml, not more than about 4 ml, not more than about 3 ml, not more than about 2 ml, not more than about 1 ml, not more than about 0.5 ml, not more than about 0.3 ml, or not more than about 0.1 ml. In some embodiments, the harvest vessel and/or sample container comprises a volume of between about 1 ml to about 100 ml, between about 10 ml to 90 ml, between about 15 ml to about 80 ml, between about 20 ml to about 70 ml, between about 25 ml to about 60 ml or between about 30 ml to about 50 ml.

In some embodiments, the harvest vessel and/or sample container of the purification device comprise a volume of about 50 ml. In some embodiments, the harvest vessel and/or sample container of the purification device comprises a volume of about 150 ml. In some embodiments, the harvest vessel and/or sample container of the purification device comprises a volume of about 200 ml.

In some embodiments, the harvest vessel and/or sample container of the purification device comprises a volume of at least about 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, about 1000 ml, about 2000 ml, about 3000 ml, about 4000 ml, about 5000 ml, about 6000 ml, about 7000 ml, about 8000 ml, about 9000 ml, about 10000 ml, about 15000 ml, about 20000 ml, about 25000 ml, about 30000 ml, about 35000 ml, about 40000 ml, about 45000 ml, or about 50000 ml. In some embodiments, the harvest vessel and/or sample container comprises a volume of not more than about 50000 ml, not more than about 45000 ml, not more than about 40000 ml, not more than about 35000 ml, not more than about 30000 ml, not more than about 25000 ml, not more than about 20000 ml, not more than about 15000 ml, not more than about 10000 ml, not more than about 9000 ml, not more than about 8000 ml, not more than about 7000 ml, not more than about 6000 ml, not more than about 5000 ml, not more than about 4000 ml, not more than about 3000 ml, not more than about 2000 ml, not more than about 1000 ml, not more than about 950 ml, not more than about 900 ml, not more than about 850 ml, not more than about 800 ml, not more than about 750 ml, not more than about 700 ml, not more than about 650 ml, not more than about 600 ml, not more than about 550 ml, not more than about 500 ml, not more than about 450 ml, not more than about 400 ml, not more than about 350 ml, not more than about 300 ml, not more than about 250 ml, not more than about 200 ml, not more than about 150 ml. In some embodiments, the harvest vessel and/or sample container comprise a volume of between about 150 ml to about 50000 ml, between about 200 ml to 45000 ml, between about 250 ml to about 40000 ml, between about 300 ml to about 35000 ml, between about 350 ml to about 30000 ml, between about 400 ml to about 25000 ml, between about 450 ml to about 20000 ml, between about 500 ml to about 15000 ml, between about 550 ml to about 10000 ml, between about 600 ml to about 9000 ml, between about 650 ml to 8000 ml, between about 700 ml to about 7000 ml, between about 750 ml to about 6000 ml, between about 800 ml to about 5000 ml, between about 850 ml to about 4000 ml, between about 900 ml to about 3000 ml, between about 950 ml to about 2000 ml or between about 1000 ml to about 1500 ml.

Preferably, the sample containers of the purification device are configured to contain a volume of 0.25 to 100 mL, more preferably from 0.25 to 500 mL, more preferably from 0.25 to 1000 mL, and more preferably from 0.25 to 1500 mL.

Alternatively, the sample containers of the purification device are configured to contain a volume of at least 50 ml, 100 ml, 150 ml, 200 ml, 250 ml, 300 ml, 350 ml, 400 ml, 450 ml, or 500 ml. In some embodiments, the sample containers of the purification device comprise at least 200 ml.

In some embodiments, the harvest vessel and/or sample container comprise a cover or lid, to prevent unwanted components from entering said harvest vessel and/or sample container (for example, RNases, microbial contamination or other degrading compounds or organisms) and from shielding the content of the harvest vessel and/or sample container from the outer environment. In some embodiments, the harvest vessel and/or sample container comprise the lid to limit exchange with the environment. In some embodiments, the lid of the harvest vessel and/or sample container is removable. In some embodiments, the lid of the harvest vessel and/or sample container is not removable. Alternatively, the harvest vessel and/or sample container are uncovered.

In some embodiments, the lid can prevent excessive water evaporation and loss of other critical volatile components. In some embodiments, the lid can prevent oxidation of the reagents or any components. In some embodiments, the lid can provide with protection from light (if needed). In some embodiments, the lid prevents contamination from any other potential chemical compound.

In some embodiments, the lid comprises at least one opening for filling, draining and sampling. In some embodiments, the at least one opening is positioned on the top of the lid.

In an embodiment the sample containers of the purification device comprise a liquid holding said compound of interest and magnetic particles.

In an embodiment, said liquid results from an IVT reaction. In another or further embodiment, said liquid contains DNA, RNA, modified RNA, polypeptides, proteins, and/or modified proteins. In yet another embodiment said liquid contains at least one reagent. In yet another embodiment said liquid contains one or more reagents used for IVT. In yet another embodiment, said liquid comprises impurities. In a further embodiment said impurities are nucleotides, enzymes, proteins, proteins, DNA templates, dsRNA, or any other by-products of IVT known in the art.

The magnetic particles can have any size suitable for binding nucleic acid, including commercially available sizes, such as a diameter ranging from about 0.3 μm to about 10 μm in diameter, e.g., about 0.3, 0.5, 1, 2, 3, 4, 5, 6, 7, 8, 9, or 10 μm in diameter, including all ranges and subranges therebetween.

In an embodiment, said magnetic particles are silica-based magnetic particles are carboxyl coated paramagnetic particles, silica-based paramagnetic particles, or combinations thereof. Silica-based magnetic particles can comprise, in some embodiments, a paramagnetic core coated with siliceous oxide, thus providing a hydrous siliceous oxide adsorptive surface to which nucleic acid can bind (e.g., a surface comprising silanol groups). The magnetic particles can, in additional embodiments, be surface-modified to produce functionalized surfaces, such as weakly or strongly positively charged, weakly or strongly negatively charged, or hydrophobic surfaces, to name a few. Alternatively, the magnetic particles may be poly styrene divinylbenzene particles, polymethacrylate particles, cross-linked agarose particles, or allyl dextran with N—N-bis acrylamide particles. It will be obvious for one skilled in the art that any material that is suitable for binding nucleic acids, proteins, or other biomolecules, may be used with the device or system as disclosed herein.

The magnetic particles of the system may be dispensed to the sample containers by one of said handling apparatuses.

The compound of interest is able to bind to said magnetic particles by means of affinity binding. In an embodiment, the compound of interest binds to the magnetic particle in the presence of a binding buffer, preferably a binding buffer containing a chaotropic agent. In the presence of the chaotropic agent, the compound of interest reversibly binds to the magnetic particle.

In another embodiment, the magnetic particle is coated with a ligand that interacts with the compound of interest.

The system as disclosed herein can be arranged in a cabinet, preferably with a unit for laminar flow generation. In an embodiment, the storage unit for storing ingredients is positioned outside said cabinet.

In an embodiment, the cabinet is designed to allow the provision of filtered, sterile air to be circulated within the units. Air filtering means may include for instance a HVAC system with HEPA filters.

The housing of the cabinet may be made of any material suitable in the art such as metal alloy, metal, or plastic. In one embodiment, a cabinet is made from a material comprising aluminum or stainless steel. In a specifically preferred embodiment, said cabinet is made of a material comprising stainless steel.

Preferably, the system and the cabinet are designed and operated that they only require limited handling of the operator. This is to avoid contamination and disturbance of the process conditions. If irregularities are observed, the operator can manipulate the process via one or more control devices present inside or outside the cabinet. These control devices control (parts of) the process taking place in the cabinet. The cabinet may be coupled to one or more control devices that are configured to perform multivariate analysis, automatically control the operation of the processes, and optionally, communicate with components remotely (using, for example, network protocols) in order to control operation in the unit(s).

Each cabinet is preferably mobile and provided with transportation means. Transportation means can include any means suitable in the art, both manually and/or electronically controlled, and include but are not limited to wheels, tracks or rolls.

In an embodiment, the system as disclosed herein comprises at least one computer processor operatively coupled to the IVT unit and/or processing unit. In an embodiment, the system comprises at least 1, 2, 3, 4, 5, 6, 7, 8, 9, or 10 computer processors. In another embodiment, the IVT unit and the processing unit are operatively coupled to the same computer processor. In another embodiment, the IVT unit and the processing unit are operatively coupled to different computer processors. In yet another embodiment the plurality of chambers and the handling apparatuses of the IVT unit are operatively coupled to different computer processors. In yet another embodiment the plurality of chambers and the handling apparatuses of the IVT unit are operatively coupled to the same computer processors. In yet another embodiment the purification device and the handling apparatuses of said device are operatively coupled to different computer processors. In yet another embodiment the purification device and the handling apparatuses of said device are operatively coupled to the same computer processors. In yet another embodiment all handling apparatuses are coupled to the same computer processor.

In some embodiments, the system disclosed herein allows for the production of RNA without requiring scale-up and ensuring fast availability in clinics. In some embodiments, said system has a small footprint while integrating all the process steps necessary for high-quality RNA production. In some embodiments, the process is optimal and adaptable for custom mRNA constructs.

### Methods

The disclosure also comprises methods for the production of RNA. In a particularly preferred embodiment, said method is executed by means of a system as described in one of the embodiments above.

In an embodiment, the IVT reaction is performed in a plurality of chambers and comprises an in vitro transcription step starting from a DNA template. The steps of the IVT may be:
(i) providing one or more IVT reagents in a first chamber of the plurality of chambers;
(ii) repeating step (i) until at least a portion of the plurality of chambers is provided with one or more IVT reagents;
(iii) performing said IVT reaction in the chambers provided with one or more IVT reagents; and
wherein the resulting IVT reaction is subsequently subjected to at least one purification step.

Without wishing to be bound by theory, the reaction conditions that need to be fulfilled in an IVT reaction are the provision of linear DNA that serves as a template that is copied into RNA by a polymerase, dNTPs, an RNA polymerase that incorporates the dNTPs, a magnesium-containing buffer that catalyzes the reaction and a specific temperature of the reaction, typically 37° C. It will be obvious to one skilled in the art that any reaction conditions that are yielding mRNA may be used with the method as described herein.

In an embodiment of the method disclosed herein, at least a part of said IVT reagents are combined into an IVT premix. In a further embodiment said IVT premix comprises at least dNTPs, template DNA, and one or more RNA polymerase buffer components. In an embodiment, the IVT reagents are stored in a storage unit, wherein the storage unit is in fluid connection with a pump system and wherein said pump system is configured to provide the desired amount of one or more IVT reagents for preparing an IVT premix to an IVT premix container. Said storage unit may be cooled to a temperature below 10° C., preferably 4° C., as previously described.

The total volume of the premix is equal or larger than the sum of the volumes of the individual IVT reactions in said chambers, in an embodiment.

In an embodiment, the IVT premix is heated to a temperature of between 35° C. to 55° C. before a portion of said premix is provided to a chamber of said plurality of chambers. In an embodiment, said IVT premix is heated to a temperature between 35° C. and 55° C., preferably between 35° C. and 50° C., 35° C. and 45° C., 35° C. and 40° C., or 35° C. and 37° C. before a portion of said premix is provided to a chamber of said plurality of chambers. Alternatively said IVT premix is heated to a temperature between 37° C. and 55° C., preferably between 40° C. and, 45° C. and 55° C. or 40 and 55° C. before a portion of said premix is provided to a chamber of said plurality of chambers.

In an embodiment, the IVT premix container is heated at a temperature of between 35° C. and 55° C., preferably between 35° C. and 50° C., 35° C. and 45° C., 35° C. and 40° C., or 35° C. and 37° C. Alternatively, the IVT premix container is heated at a temperature of between 37° C. and 55° C., preferably between 40° C. and, 45° C. and 55° C. or 40 and 55° C. In a preferred embodiment, the premix container is heated at 37° C.

In an embodiment of the method disclosed herein, an RNA polymerase is added to at least one chamber of said plurality of chambers. In other embodiments, a plurality of RNA polymerases is added to the chamber, preferably 2, 3, 4, 5, 6, 7, 8, 9, or 10 RNA polymerases. In a further embodiment, said RNA polymerase is added before or after said chamber received a portion of said IVT premix. Combining the IVT premix with the RNA polymerase triggers the start of the reaction. In some embodiments, it is desired that the premix is heated in the absence of the RNA polymerase and thus the start of the reaction is delayed until the premix and the RNA polymerase are combined in the plurality of chambers. In other embodiments, said RNA polymerase is part of said IVT premix. The RNA polymerase used is as previously described.

In some embodiments of the method, the IVT reagents comprise a capping reagent for co-transcriptional capping. Non limiting examples of co-transcriptional capping reagents include CleanCap (TriLink) or ARCA (CellScript). Alternatively, capping occurs post-transcriptional using any cap analog structure known in the art.

Preferably, the system as disclosed herein is used for purifying an mRNA molecule from an IVT process, or for purifying during pre- and/or post-capping. In an embodiment, when the mRNA molecule is co-transcriptional capped, said mRNA is purified after the capping. In another embodiment, when the mRNA is post-transcriptionally capped, the purification step is done prior to capping. In yet another or further embodiment, purification is done post capping. In yet another embodiment, a purification step is done before capping and a second purification step is done post capping. The IVT resulting product contains besides the desired mRNA product, an array of reaction by-products such as salts, nucleotides, enzymes, proteins, DNA templates, or dsRNA. These can interfere with the capping process, and reduce the transaction efficiency and overall purity of the final product. Enzymatic capping immediately following IVT, without intermediate treatment of the reaction product, produces reduced amounts or approaching 0% capped mRNA molecules. The system disclosed herein is designed to perform mRNA purification with high precision, in an automated manner under GMP-compliant conditions, and is adaptable to perform the purification upstream and/or downstream capping. The system allows for a continuous production of small or medium volumes of the compound of interest.

In some embodiments of the method, a finalized IVT reaction from a chamber of said plurality of chambers is transferred to an intermediate vessel. In a further embodiment, the IVT reactions of said plurality of chambers are pooled in said intermediate vessel. In yet a further embodiment, the transfer and pooling of the IVT reaction is done by means of a robotic arm.

The IVT reaction is terminated by means of the addition of at least a DNase in an embodiment of the method. In another embodiment, the IVT reaction is terminated by the addition of EDTA. In yet another embodiment, both a DNase and/or EDTA are added to terminate the IVT reaction. The DNase enzymatically digests the DNA template and thus ends the IVT reaction while the EDTA is a chelating agent that depletes the reaction of the Mg²⁺ ions which also leads to the IVT reaction being terminated.

In an embodiment, the DNase and/or EDTA is added in the plurality of chambers. In another embodiment, said DNase and/or EDTA is added to the intermediate vessel. In a further embodiment, the DNase and/or EDTA is added by means of a pump, injector, or robotic arm.

In an embodiment, at least one purification step after IVT makes use of magnetic particles able to bind to said RNA molecules. In a further embodiment, the IVT reaction comprising RNA molecules or a portion of said IVT reaction is provided to one or more sample containers positioned in a sample holder. In a further embodiment said magnetic particles are added to said sample container. Alternatively, the sample container comprises magnetic particles and the IVT reaction comprising RNA molecules or a portion of said IVT reaction is provided to said container.

In a preferred embodiment of the at least one purification step, a portion of said IVT reaction is provided by the handling means to the container and purified.

In some embodiments of the method, the RNA binds to the magnetic particles and subsequently, the magnetic particles are captured or induced to move towards a magnet unit present in the vicinity of the sample holder, thereby causing a separation of the RNA molecules bound to said magnetic particles and any remaining IVT liquid. In some embodiments, a binding buffer is used to mediate the reversible binding between the compound of interest and the magnetic particles. In a further embodiment said binding buffer can comprise a chaotropic agent, alcohol, a PEG, a salt, or a mixture thereof. Said chaotropic agent can be chosen from guanidine salts, such as hydrochloride (GuHCl) and guanidium thiocyanate (GuSCN); lithium salts, such as lithium acetate and lithium perchlorate; or sodium salts such as NaCl and combinations thereof. In an embodiment, said binding buffers are devoid of chaotropic agents.

The alcohol can be chosen from isopropanol, ethanol, methanol, butanol, and combinations thereof. In an embodiment, said alcohol is present at a concentration of 10% to 50%, from 10% to 40%, from 10% to 30%, from 10% to 20, from 15% to 20% v/v, including all ranges and subranges therebetween.

In an embodiment, said binding buffer may comprise PEG, either as alternative to the alcohol or in combination with said alcohol. The concentration of PEG in the binding buffer can range from 10% to 40%, from 20 to 40%, from 20% to 35%, from 20% to 30% or from 25% to 35%, including all ranges and subranges therebetween. In an embodiment, 30% PEG is used. In an embodiment, said PEG used in the binding buffer is chosen from PEG 600, PEG 1000, PEG 2000, PEG 3000, PEG 4000, PEG 6000, PEG 8000, PEG 10.000, PEG 20.000. In an embodiment, used PEG is PEG 8000.

The at least one salt can be present in the binding buffer in a concentration ranging from 0.1M to 5 M, for example, from 0.1 to 4M, from 0.1M to 3M, from 0.1M to 2M, from 0.1M to 1M, from 0.5 to 1M, from 0.5 to 2M, from 1M to 2M and from 2M and 3M and from 3M to 5M, including all ranges and subranges therebetween. According to various embodiments, the at least one salt can be sodium chloride (NaCl).

In an embodiment, the binding buffer comprises Tris-HCl, NaCl, EDTA, and ethanol.

In embodiments, said binding buffer can have a pH ranging from 5 to 10, such as from 5 to 9, from 5.5 to 8.5, from 6 to 8, or from 6.4 to 7.5 and all ranges and subranges therein between. In embodiments, said binding buffer comprise of at least one first alcohol and/or PEG, at least one salt and at least one optional chelating agent such as EDTA

The magnetic particle can be present in the binding buffer in a concentration ranging, for instance, from about 0.1 μg/μl to about 60 μg/μl, such as from 0.5 μg to about 60 μg/μl, from about 0.75 μg/μl to about 55 μg/μl, from about 1 μg/μl to about 50 μg/μl, from about 2 μg/μl to about 45 μg/μl, from about 3 μg/μl to about 40 μg/μl, from about 4 μg/μl to about 35 μg/μl, from about 5 μg/μl to about 30 μg/μl, from about 6 μg/μl to about 25 μg/μl, from about 7 μg/μl to about 20 μg/μl, from about 8 μg/μl to about 15 μg/μl, or from about 9 μg/μl to about 10 μg/μl, including all ranges and subranges therebetween. By way of non-limiting embodiment, the at least one magnetic particle may be chosen from Qbeads and may be present in the binding buffer B1 in a concentration ranging from about 0.5 μg/μl to about 5 μg/μl. In alternative embodiments, the at least one magnetic particle may be chosen from Grace beads and may be present in the binding buffer B1 in a concentration ranging from about 2 μg/μl to about 60 μg/μl.

In embodiments of the methods as taught herein, a volumetric ratio between the sample and the binding buffer can range, for example, from 1:1 to 1:3, such as from 1:1 to 1:1.5, or from 1:1.5 to about 1:2.5, including all ranges and subranges therebetween. Incubation time period for the mixed solution comprising sample comprising at least one nucleic acid of interest, binding buffer and silica-based magnetic particles can range from 0.1 minute to 30 minutes, from 0.1 minutes to 25 minutes, from 0.1 minutes to 20 min, from 0.1 to 10 minutes, or from 0.1 to 5 minutes, from 0.1 to 2 minutes including all ranges and subranges therebetween.

In a further embodiment, the remaining IVT liquid is removed after the separation of the RNA molecules from the remaining IVT liquid.

In yet a further embodiment, after removal of the remaining liquid, a further liquid is added to said RNA bound to magnetic beads and the separation step is repeated.

The sample holder is subjected in some embodiments of the method to a mechanical motion, thereby allowing mixing of said IVT reaction or further liquid with said magnetic particles.

In some embodiments of the method, the sample holder with a sample container rotates between or after a capturing step to at least one subsequent position.

The sample holders with containers are positioned on a base portion of a sample plate and said sample plate is able to rotate. Rotation of the sample plate allows the delivery of the sample container to different dispensers such as injectors, robotic arms or pumps, where a component or a liquid can be added or removed from the sample container.

In other embodiments of the method, the sample plate rotates between or after a capturing method step, thereby moving the sample holder with a sample container to a subsequent position.

In alternative embodiments of the methods disclosed herein, the sample plate does not move between the capturing steps, thereby allowing the sample holders to remain in a fixed position.

In some embodiments of the method, the magnetic particles are added to the sample container by means of an injector or a robotic arm provided with one or more nozzles, needles, and/or tips, as previously described. In further embodiments, the addition and/or removal of a liquid occurs by means of one or more injectors, or robotic arms provided with one or more nozzles, needles, and/or tips, as previously described.

In an embodiment, the mechanical motion of the sample holder that allows the RNA to mix with the magnetic particles is shaking or agitating. The shaking or agitating of the sample holder causes the motion of the magnetic particles which become suspended in the liquid medium and come in contact with the compound of interest.

The magnet unit comprises a permanent magnet in an embodiment of the method disclosed herein. The mechanical motion prevents capturing or introducing a movement of the magnetic particles towards said magnet unit.

In a particularly preferred embodiment, the magnetic particles used in the method disclosed herein are silica based magnetic particles, as previously described. In an embodiment of the method, a binding buffer as described above, is used for mediating binding of the RNA to said magnetic particles.

Upon binding the nucleic acid to the magnetic particles and after separation of the modified magnetic particles using a magnet, the magnetic particles may be in some embodiments, combined, rinsed, or washed with one or more wash buffers. A wash buffer can comprise, for example, at least one alcohol and optionally at least one salt. The modified magnetic particles can be rinsed once or multiple times with the wash buffer, and any additional washing can employ the same or different compositions, concentrations, and/or volumetric amounts. According to various embodiments, the at least one alcohol can be chosen from isopropanol, methanol, ethanol, butanol, and combinations thereof. According to various embodiments, the at least one salt can be chosen from ammonium sulfate ((NH4)2SO4), ammonium acetate (NH4Ac), lithium acetate (LiAc), potassium acetate (KAc), sodium acetate (NaAc), sodium chloride (NaCl), and combinations thereof.

In embodiments of the methods as taught herein, said wash buffer comprises at least a second alcohol in a concentration ranging, from 50% to 100% by volume/volume (v/v), from 55% to 95%, from 60% to 85%, or from 60% to 80% by v/v, including all ranges and subranges therebetween. In embodiments, the second alcohol can be chosen from isopropanol, methanol, ethanol, butanol, and combinations thereof. The second alcohol in the wash buffer can be the same or different from the first alcohol in the binding buffer. In some embodiments, the second alcohol can be ethanol. In non-limiting embodiments, the wash buffer optionally has at least one salt. The optional salt, if present, in the binding buffer can is in a concentration ranging from 0.1 M to 5 M, for example, from 0.3 M to 4 M, from 0.1 M to 3 M, from 0.1 M to 2 M, from 0.1 M to 1 M, and from 1 M to 2 M, including all ranges and subranges therebetween. According to various embodiments, the salt in the wash buffer can be sodium chloride (NaCl). In some embodiments, the modified magnetic particles can be washed one or more time with at least one of said wash buffer. For example, said modified magnetic particles may be washed once, twice, or more with the wash buffer with intervals of separation of the modified magnetic particles by using a magnet in between the washes.

In some embodiments, the modified magnetic particles are rinsed once or multiple times with said wash buffer, and additional washing steps may be employed with for instance the same or different compositions, concentrations, and/or volumetric amounts.

After the addition and removal of the wash buffer, the modified magnetic particles with the compound of interest reversibly bound to the surface are substantially free of contaminants such as salts, proteins, enzymes, etc. According to various embodiments, the modified magnetic particles thus produced can then be incubated with one or more elution buffers to release the bound compound of interest and separate it from the magnetic particles.

According to various embodiments, the elution buffer is a low conductivity solution wherein the conductivity of the buffer ranges from 0.001 to 40 mS/cm, more preferably from 0.01 to 40 mS/cm, from 0.1 to 40 mS/cm, from 0.5 to 40 mS/cm, more preferably from 0.5 to 30 mS/cm, from 0.5 to 20 mS/cm, from 0.5 to 10 mS/cm, including all ranges and subranges therebetween.

In another or further embodiment, said elution buffer comprises a salt concentration of between 0.01 and 50 mM, more preferably between 0.1 to 40 mM, more preferably between 0.1 and 30 mM, more preferably between 0.1 and 20 mM. Possible salts include sodium citrate, sodium chloride, sodium phosphate, potassium chloride, potassium phosphate and combinations thereof.

The pH of the elution buffer can range, for example, from 5 to about 10, such as from 5.5 to about 9, from 6 to 8, or from 6.4 to about 7.5, including all ranges and subranges therebetween.

For example, in some cases elution buffer can comprise water; in others water and EDTA, or only Tris, or Tris and EDTA, or Sodium citrate, or phosphate buffer, or Phosphate-buffered saline (PBS). The concentration of the sodium citrate, if used as elution buffer can range from 0.5 mM to 10 mM, for example from 0.6 mM to 5 mM, from 1 mM to 2 mM, including all ranges and subranges therebetween. The pH of the Sodium citrate, if used as elution buffer can range from pH 5.4 to 7.5, from pH 6 to pH 7, from pH 6 to 6 pH 6.5, including all ranges and subranges therebetween. According to non-limiting embodiments, the elution buffer can comprise water or 10 mM Tris-HCl, 1 mM EDTA, pH 7.4, or 10 mM Tris-HCl, pH 7.4, or 1 mM citrate Na, pH 6.4.

In embodiments, said elution buffer is devoid of toxic chaotropic agents such as guanidine salts (guanidinium thiocyanate or guanidine thiocyanate), iodide, perchlorate and trichloroacetate, preferably guanidine salts.

In embodiments, at least one modified particle incubated with the elution buffer for a time period ranging from 30 seconds to 30 minutes, from 1 minute to 20 minutes, from 1 minute to 10 minutes including all the ranges and subranges therebetween.

In an embodiment, upon adding said elution buffer, the sample holder is subjected to a mechanical motioning, thereby allowing mixing of said magnetic particles with the elution buffer. Once the motioning stops, the magnetic particles move towards said magnet unit, thereby causing a separation of the elution buffer comprising the compound of interest and the magnetic particles. In a further embodiment, the elution buffer comprising the RNA is removed from said sample container and stored in a harvest vessel.

The purification step includes or precedes a post-transcriptional capping step in some embodiments of the method disclosed herein. In an embodiment, the purification step is part of a pre-capping step and is followed by a post-transcriptional capping step.

In some embodiments, after capping, a second purification step is performed. In a further embodiment, said second purification step makes use of magnetic particles able to bind to said capped RNA molecules. The second purification step is executed as previously described for the first purification step.

Said second purification step may be performed using the same purification device. In other embodiments, the second purification step is performed in a second purification device. The two purification devices may be placed adjacent to one another or in a nested configuration.

In an embodiment, the system as disclosed herein is used in conjunction with dead-end filtration. The dead-end filtration of the mRNA may be performed after the IVT reaction and before loading on the purification device. In another embodiment, the dead-end filtration is performed after the mRNA is purified with the purification device disclosed herein.

Alternatively, any filtration method may be used with the system disclosed herein, such as, but not limited to diafiltration or tangential flow filtration (TFF).

The method as disclosed herein is executed by means of a device or system according to embodiments previously described. In an embodiment, the method is semi- or fully automated. The method is rapid and efficient while reproducing small-scale repetitive manual procedures. Said method is compliant with GMP conditions, is not prone to human error, and delivers a product of high purity.

The present disclosure will now be further exemplified with reference to the following examples. The present disclosure is in no way limited to the given examples or to the embodiments presented in the figures.

### Figures

The present disclosure is in no way limited to the embodiments described in the examples and/or shown in the figures. On the contrary, methods according to the present disclosure may be realized in many different ways without departing from the scope of the disclosure.

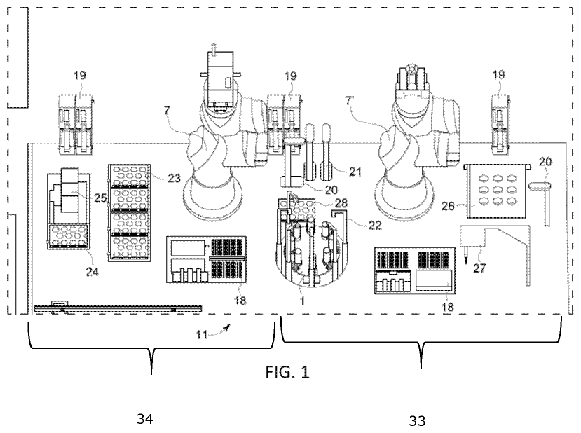

FIG. 1. shows an embodiment of a system for producing RNA (11) comprising an IVT unit (34) and a downstream processing unit (33) and handling apparatuses: robotic arms (7, 7′), injectors (20, 21, 22, 27), and pumps (19). The robotic arms (7, 7′) make use of needles and/or tips (18). In an embodiment, the compound of interest obtained using the method disclosed herein is further downstream processed in a container (26).

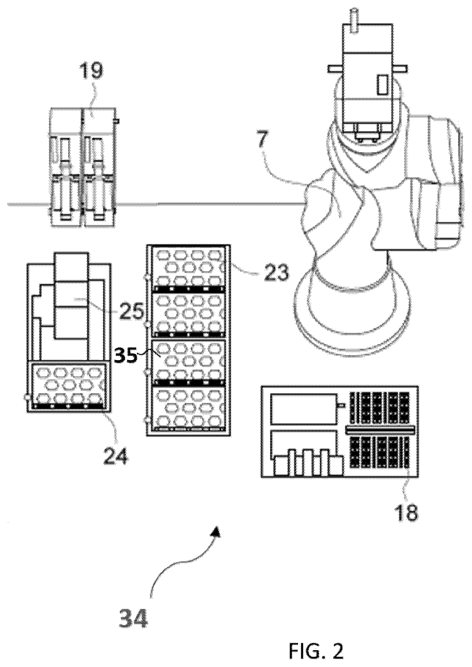

The IVT unit (34) is depicted in FIG. 2. In an embodiment, a desired amount of reagents for preparing an IVT premix is delivered to an IVT premix container (24). The IVT unit (34) comprises a heating unit (25) for heating the IVT premix container (24) at 37° C. The premix contains in some embodiments dNTPs, template DNA, and one of more RNA polymerase buffer components. In an embodiment, the heating is done before the addition of the RNA polymerase. In another embodiment, the RNA polymerase is also added to the IVT premix container (24) and is heated.

In a preferred embodiment, the IVT premix at 37° C. is transferred to a plurality of chambers (23) where the IVT reaction is executed in the presence of RNA polymerase, a plurality of RNA molecules being obtained.

The IVT unit (34) comprises handling apparatuses, such as a robotic arm (7), injectors (20, 21, 22, 27), and/or pumps (19) configured for dispensing and/or removing a reagent, a reagent mixture or a liquid in the premix container (24) and plurality of chambers (23) arranges in cartridges (35). In the example of FIG. 2, the IVT unit (34) has 4 cartridges (35).

The RNA molecules are further purified in the downstream processing unit (33) that contains a purification device (1).

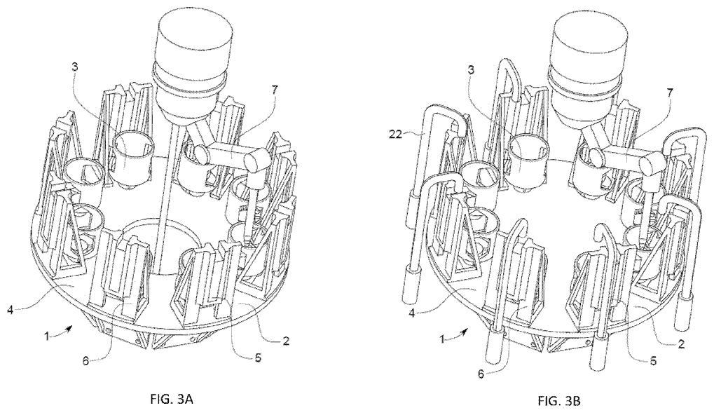

A purification device (1) for separating and/or purifying an RNA molecule obtained by IVT according to an embodiment of the current specification is illustrated in FIG. 3 wherein the purification device is in the form of a carousel. In some embodiments, as shown in FIGS. 1 and 3, the device (1) may be configured to to rotate around an axis. Alternatively, the purification device can be fixed and therefore cannot rotate (not shown). According to the particular embodiment illustrated in FIG. 3, the device (1) comprises a sample plate (2) equipped with sample holders (3) disposed on the base portion (4) of the sample plate (2). In the embodiment of FIG. 3 the sample plate (2) comprises eight sample holders (3). It will however be apparent to the skilled person that this number is flexible. Hence, other embodiments are contemplated wherein a different number of sample holders (3) are provided. Sample containers (8) comprising the compound of interest in a liquid medium are positioned inside the sample holders (3). It will also be apparent to the skilled person that the sample plate can have many forms. In some embodiments, may not be round as shown in FIGS. 1 and 3. In another embodiment, the purification device (1) does not comprise a sample plate (2), but comprises one or more sample holders (3) present in said system (not shown in the figures).

A magnet unit (5) is positioned at each sample holder (3). The magnet unit (5) as shown in the embodiment of FIG. 3A and FIG. 3B is arranged in a housing (6) that is open at the side facing the sample holder (3). In some embodiments, the magnet unit (5) comprises a permanent magnet, in other embodiments comprises a temporary magnet or an electromagnet.

The sample holders (3) can perform mechanical motions that in some embodiments are rotations around their axis and in other embodiments are shaking motions. The sample holders (3) may move independently from one another or may synchronize their motions. In the example shown in FIG. 3 the motion is driven by a motor unit or a shaker unit. The sample plate (2) is configured to rotate around an axis clockwise and counterclockwise.

In the embodiment shown in FIG. 3A, a robotic arm (7) is positioned in the centrum of the sample plate (2). In the embodiment of FIG. 3B, the robotic arm (7) is positioned outside the sample plate (2) or the device (1). The robotic arm (7), equipped with a nozzle tool (13), a pipetting tool (17), needles and/or tips, is configured for dispensing and/or removing components and/or liquids from the sample containers (8).

In an embodiment, the RNA to be purified is produced by an IVT reaction. A sample containing said RNA suspended in a reaction medium is dispensed in a sample container (8) by a robotic arm (7). Magnetic particles (9) are also dispensed in the sample container (8) by the robotic arm (7).

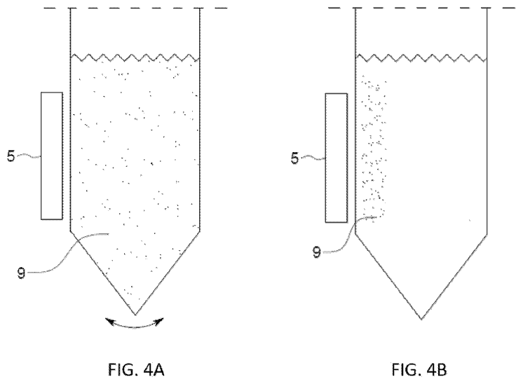

Capturing of the magnetic particles (9) is shown in FIG. 4A and FIG. 4B. The magnet unit (5) is configured to attract and capture said particles (9) residing in the sample container (8). The motion of the sample holder (3) drives the captured or free status of the silica beads (9). When the sample holder (3) is in motion, the magnetic particles (9) are free to move in the liquid and interact with the RNA molecules (FIG. 4A). The RNA molecules reversibly bind to said silica beads (9). When the sample holder (3) stops its motion, the silica beads (9) become captured (FIG. 4B). In an embodiment where an electromagnet is used, the captured or free status of the magnetic particles is controlled by activating and deactivating the electric current.

While the magnetic particles (9) are captured inside the sample container (8), the liquid content of said container can be removed or replaced without losing the magnetic particles (9) or RNA bound to them. Dispensing and removing of liquids in the sample container is performed by handling apparatuses such as robotic arms (7), injectors (20, 21, 22, 27), or pumps (19). The robotic arm (7) can move at any random location and can access any sample container (8). The injectors (20, 21, 22, 27) or pumps (19) are located at fixed positions and the sample containers (8) are delivered to them by the rotation of the sample plate (2). The robotic arm (8) and the other handling apparatuses are connected to one or more reagent storage (29), waste vessels, and harvest vessels (28).

In an embodiment, the purification device (1) is designed for executing one or more steps of RNA purification: binding of the RNA to magnetic particles in the presence of a chaotropic agent, washing of the silica beads bound RNA, elution of the RNA, and collection of the purified RNA in a harvest vessel. This allows for performing multiple automated and precise washing steps, without losing the RNA. The device is configured to allow repetitive separation and/or purification of RNA and can be operated continuously. In a preferred embodiment, the magnetic particles are silica-based magnetic beads.

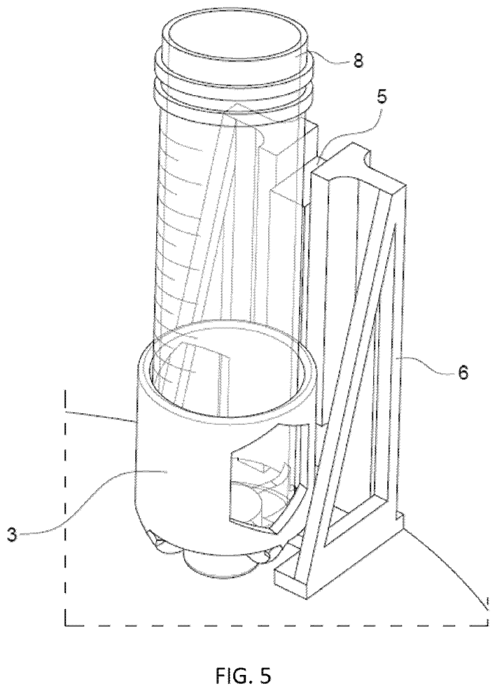

In FIG. 5 an embodiment of the sample holder (3) with a sample container (8) having a capacity of 50 ml, is shown. In some embodiments, the volume may vary, depending on the process. Said sample container (8) if fitted directly in the sample holder (3). It will be apparent that also other sample containers can be used in the context of the current disclosure.

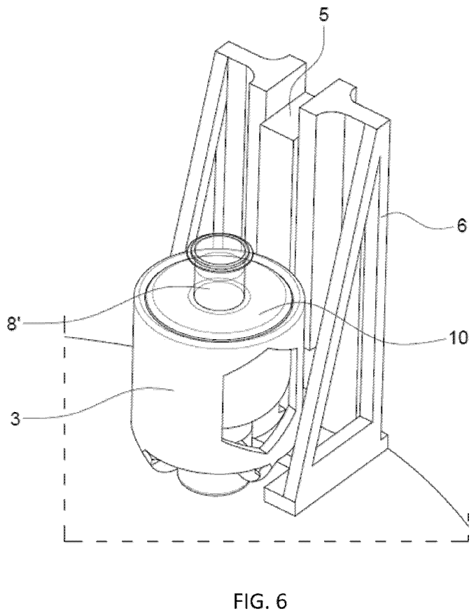

FIG. 6 shows an embodiment of a sample holder (3) with a sample container (8′) having a capacity of 2 ml. Said sample container (8′) is fitted in an adaptor (10) that adapts the sample holder (3) for the size of the sample container (8′). In other embodiments, sample containers (8′) of other sizes and volumes are used.

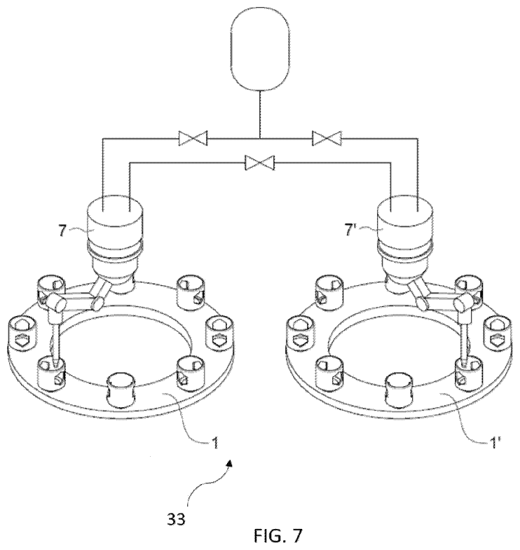

A particular embodiment of the downstream processing unit (33) is shown in FIG. 7, wherein said downstream processing unit (33) comprises two purification devices (1 and 1′) and two robotic arms (7 and 7′) are provided. In this embodiment, said purification devices are positioned adjacent to each other. This configuration allows for a higher capacity of production while maintaining the footprint of the purification devices to a minimum. Alternatively, this configuration can be used for different purification processes. For instance, a first purification device (1) can be used for pre-capping purification, whereas a second purification device (1′) can be used in a post-capping process. The capping reaction can take place in the final position of the first purification device (1), or in an intermediate vessel positioned downstream of the first purification device (1) and upstream of the second purification device (1′).

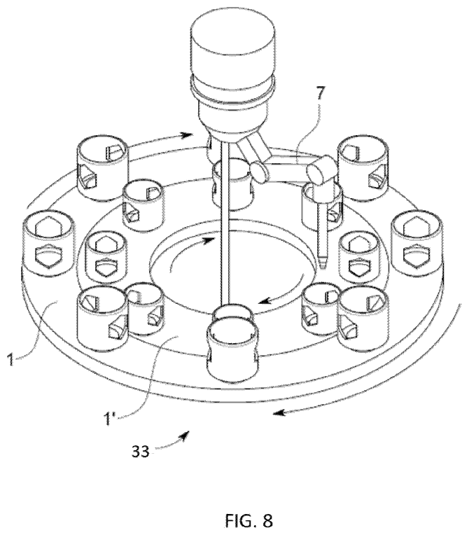

An alternative embodiment is provided in FIG. 8, wherein two purification devices (1 and 1′) are in a nested configuration and a single robotic arm (7) is provided.

A multi-purification device system as depicted in FIG. 7 and FIG. 8, allows for the performance of multiple steps concomitant such that different stages of the separating and/or purifying can be performed at the same time.

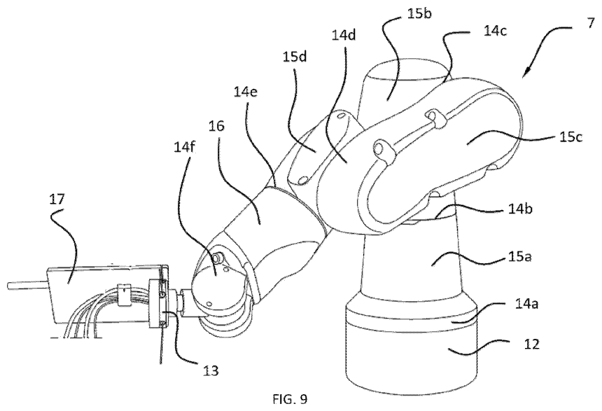

The robotic arm (7) for handling liquid media and sample containers from/to a recipient according to an embodiment as described herein is illustrated in FIG. 9. Robotic arms (7) are used for manipulation steps that take place in both the IVT unit (34) and downstream processing unit (33). One or more robotic arms (7, 7′) can be used with the system (11).

The robotic arm shown in FIG. 9 comprises a base (12), a pipetting tool (17), and a nozzle tool (13) configured to handle one or more liquid media. The nozzle tool (13) is positioned at a distal end of the robotic arm (7).

In what follows, the robotic arm (7) is described as being mounted on a horizontal surface. Other modes of installation are of course possible, and the adaptation of what follows to such other modes of installation, fall within the scope of the skilled person's abilities, and are considered to form part of the scope of the embodiments as described in the present application. For instance, the robotic arm (7) can be mounted on a vertical surface, resulting in a rotation of 90° for all subsequent orientations.

The robotic arm (7) is an articulated robotic arm (7) comprising joints (14) and wherein the robotic arm 1 is manufactured by sequentially connecting these joint (14) by multiple links (15). The robotic arm (7) as shown in FIG. 7 comprises six joints (14), allowing movement in six degrees of freedom. More specifically, in a first joint (14a), a base and a proximal end portion of a first link (15a) are connected so as to be rotatable around an axis extending in the vertical direction. The first joint (14a) is a twisting joint.

In a second joint (14b), a distal end portion of the first link (15a) and a proximal end portion of a second link (15b) are connected so as to be rotatable around an axis extending in the horizontal direction. The second joint (14b) is a revolving joint.

In a third joint (14c), a distal end portion of the second link (15b) and a proximal end portion of a third link (15c) are connected so as to be rotatable around an axis extending in the horizontal direction, in this case, parallel to the axis for the second link (15b). It should be noted that deviations from the horizontality of this third axis are possible, but the third axis will always have at least a horizontal component, it being fully horizontal being the most efficient version. The third joint (14c) is in this case a revolving joint.

In a fourth joint (14d), a distal end portion of the third link (15c) and a proximal end portion of a fourth link (15d) are connected so as to be rotatable around an axis in the longitudinal direction of the fourth link (15d). The fourth joint (14d) is a twisting joint.

In a fifth joint (14e), a distal end portion of the fourth link (15d) and a proximal end portion of a connector (16) are connected so as to be rotatable around an axis orthogonal to the fourth axis. The fifth joint (14e) is a revolving joint but it should be noted that this joint can be easily adapted to a twisting joint or rotational joint.

In a sixth joint (14f), a distal end portion of the connector (16) and a proximal end of the nozzle tool (13) are connected so as to be rotatable in a plane orthogonal to the longitudinal direction the connector (16).

Each of the joints (14) is provided with a drive motor as an example of an actuator for relatively rotating the two members connected by the joint (14). The drive motor is, for example, a servo motor which is servo-controlled via a servo amplifier by a control signal transmitted from the controller. In addition, each of the joints is provided with a rotation angle sensor for detecting the rotation angle of the drive motor and a current sensor for detecting the current of the drive motor.

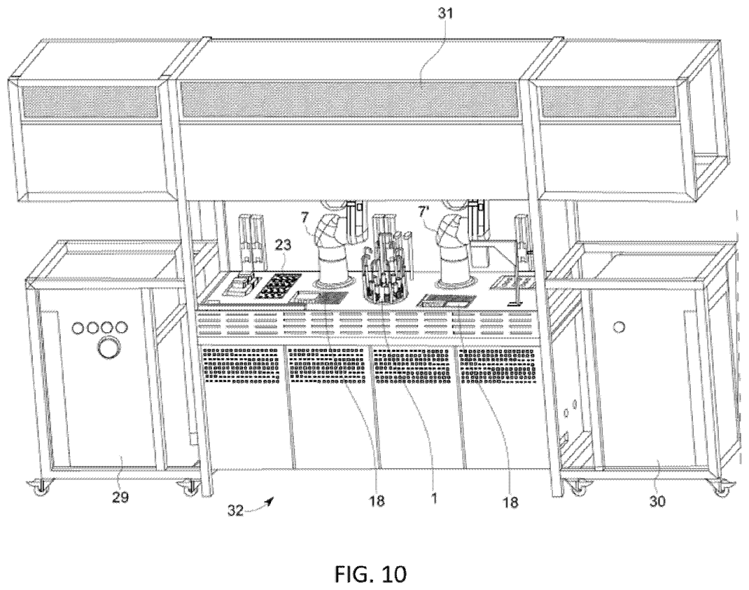

In an embodiment, the system (11) is arranged in a cabinet (32), preferably with a unit for laminar flow generation, as depicted in FIG. 10. The cabinet (32) includes a reactive storage unit (29) that in some embodiments is cooled up to 4° C., a unit for storing the compound of interest after processing (30) (if needed, cooled), and a filter unit (31). The filter unit (31) allows the provision of filtered, sterile air to be circulated within the cabinet (32). Air filtering means may include in some embodiments, a HVAC system with HEPA filters. In an embodiment, the cabinet or parts of the cabinet can have wheels, such that it can easily be transported.

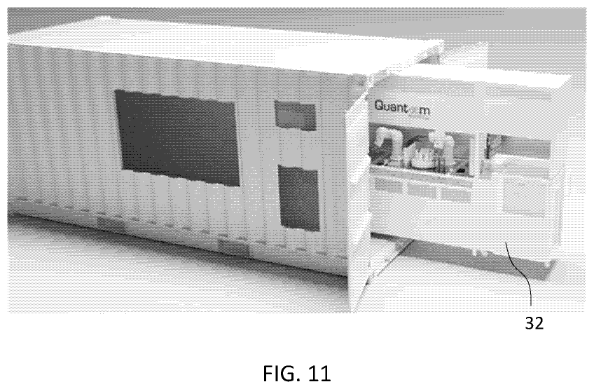

The system can be designed such that the cabinet (32) containing the system fits in a transport container as shown in FIG. 11 or in small cleanrooms. As such, it can be suitable for building modular, mobile laboratories, having multiple systems, thereby allowing the production of large amounts of RNA.

### FIGURE NUMBERS

1, 1′: purification device
2: sample plate
3: sample holders
4: base portion of the sample plate
5: magnet unit
6: housing
7, 7′: robotic arm
8, 8′: sample container
9: magnetic particles
10: adaptor
11: system
12: base of the robotic arm
13: nozzle tool
14a-f: joints
15a-e: links
16: connector
17: pipetting tool
18: needles or tips
19: syringe pumps
20, 21, 22, 27: injectors
23: plurality of chambers
24: IVT premix container
25: heating unit
26: container for downstream processing
28: harvest vessel
29: reagent storage unit
30: compound storage unit
31: filter unit
32: cabinet
33: downstream processing unit
34: IVT unit
35: cartridge

1-99. (canceled)

100. A system for producing RNA, said system comprising:
an in vitro transcription (IVT) unit for producing a plurality of RNA molecules, wherein said IVT unit comprises:
a plurality of chambers, wherein a chamber is configured to receive one or more IVT reagents and to execute an IVT reaction based on a DNA template to produce said plurality of RNA molecules; and
a processing unit for processing said plurality of RNA molecules subsequent to producing said plurality of RNA molecules,
wherein the processing unit comprises:
one or more containers, wherein a container is configured to hold a liquid medium comprising said plurality of RNA molecules and a plurality of magnetic particles; and
one or more magnet units, wherein a magnet unit is configured to capture or to introduce a movement of said plurality of magnetic particles in said container.

101. The system according to claim 100, further comprises one or more sample holders, wherein a sample holder is configured to receive a container.

102. The system according to claim 100, wherein the container is configured to perform a mechanical motion to mix said plurality of magnetic particles within said liquid medium.

103. The system according to claim 100, wherein said processing unit comprises a purification device for purifying said plurality of RNA molecules.

104. The system according to claim 100, wherein the system comprises an intermediate vessel configured for collecting and storing a finalized IVT reaction product from said plurality of chambers.

105. The system according to claim 104, wherein a portion of the finalized IVT reaction product is transferred by a handling apparatus to a container of said purification device.

106. The system according to claim 100, wherein said IVT unit is housed in a cartridge.

107. The system according to claim 100, wherein said system comprises at least one handling apparatus configured for dispensing and/or removing a reagent, a reagent mixture or a liquid.

108. The system according to claim 107, further comprising a control system configured to control dispensing and/or removing of a reagent, reagent mixture or liquid by said handling apparatuses.

109. The system according to claim 100, wherein the processing unit comprises at least one handling apparatus configured for dispensing and/or removing a component or a liquid from a sample container present in a sample holder of said purification device.

110. The system according to claim 100, said system comprising one or more sample containers.

111. A method for the production of RNA, said method comprises an in vitro transcription step starting from a DNA template, wherein the IVT reaction is performed in a plurality of chambers, said in vitro transcription step comprising:
providing one or more IVT reagents in a first chamber of the plurality of chambers;
repeating step (i) until at least a portion of the plurality of chambers is provided with one or more IVT reagents;
performing said IVT reaction in the chambers provided with one or more IVT reagents; and
wherein the resulting IVT reaction is subsequently subjected to at least one purification step.

112. The method according to claim 111, wherein the at least one purification step after IVT makes use of magnetic particles able to bind to said RNA molecules.

113. The method according to claim 111, wherein a finalized IVT reaction from a chamber of said plurality of chambers is transferred to an intermediate vessel.

114. The method according to claim 113, wherein a portion of the finalized IVT reaction from the intermediate vessel is subjected to at least one purification step.

115. The method according to claim 111, wherein at least a part of said IVT reagents are combined into an IVT premix.

116. The method according to claim 115, wherein said IVT premix is heated to a temperature of between 35° C. to 55° C. before a portion of said premix is provided to a chamber of said plurality of chambers.

117. The method according to claim 111, wherein an RNA polymerase is added to at least one chamber of said plurality of chambers.

118. The method according to claim 112, wherein said IVT reaction comprising RNA molecules or a portion of said IVT reaction is provided to one or more sample containers, wherein said magnetic particles are added to said sample container.

119. The method according to claim 118, wherein after removal of the remaining liquid, a further liquid is added to said RNA bound to magnetic beads and the separation step is repeated.
